(19) United States
(12) Patent Application Publication
(10) Pub. No.: US 2026/0014059 A1
(43) Pub. Date: Jan. 15, 2026

(54) DENTAL CLEANING TABLET

(71) Applicants: Bryson Barney, Mapleton, UT (US); Gwen Evenstad, Sandy, UT (US)

(72) Inventors: Bryson Barney, Mapleton, UT (US); Gwen Evenstad, Sandy, UT (US)

(21) Appl. No.: 19/269,566

(22) Filed: Jul. 15, 2025

Related U.S. Application Data
(60) Provisional application No. 63/671,536, filed on Jul. 15, 2024.

(51) Int. Cl. A61K 8/02; A61Q 11/00

(52) U.S. Cl. CPC A61K 8/0233; A61Q 11/00; A61K 2800/28; A61K 2800/92

(57) ABSTRACT

A dental cleaning tablet. The dental cleaning tablet includes a core, wherein the core includes one or more dental hygiene products and an outer shell, wherein the outer shell completely covers the core.

### CROSS-REFERENCE TO RELATED APPLICATIONS

This application claims the benefit of and priority to U.S. Provisional Patent Application Ser. No. 63/671,536 filed on Jul. 15, 2024, which application is incorporated herein by reference in its entirety.

### BACKGROUND OF THE INVENTION

An oral cavity or mouth, which is a main organ for breathing and food intake, is constantly exposed to external antigens through air and food, and therefore, if the oral cavity is not kept clean through brushing, etc., a dental plaque is created at the periphery of teeth and gums (gingiva), and the created dental plaque is gradually calcified to form a dental calculus on a surface of the tooth. The dental plaque and calculus have very important pathological significance in that they become a proliferation point of oral bacteria and cause gum inflammatory diseases such as gingivitis and periodontitis.

Typically, oral hygiene is maintained through regular brushing with toothpaste and/or rinsing with mouthwash. Regular use of such toothpastes and mouthwashes can reduce the growth of dental plaque, decrease the risk of developing gum disease, and prevent tooth decay. While these oral care products have been traditionally used, these products suffer from drawbacks. In particular, there is a market need for products that can be used on the go, or that are designed for easier use with children. However, the types of oral care products which address these needs are limited in number. Many toothpastes on the market are in paste or gel forms and packaged in a plastic tube. This causes an unnecessary package waste and makes a toothpaste mess when used by children that are still learning oral hygiene practices. Tubes are also difficult for children and people with dexterity challenges to use. Squeezing the toothpaste out of the tube onto a toothbrush often results in the wrong toothpaste dose for children and often causes a mess as the operation requires dexterity with 2 hands and good alignment onto the brush. Toothpaste dispensing tubes also are problematic for efficient dispensing over the product's useful lifetime. Children are often not strong enough to efficiently push the toothpaste out of the tube in a way that gets all the remaining toothpaste to the front near the tube's orifice. To get the paste out, children twist, crunch, or mash the tube, which results in wasted toothpaste and uneven distribution.

Currently, a variety of toothpastes are used in a tube form, but they are bulky and can be quite messy. It is difficult to get the last bit of paste out of the tube and it can be easily punctured by any sharp means. The tube cap often times does not screw or flip back on correctly because it is cover with dried toothpaste after several uses. If left without the cap, the paste can find a way to migrate from the tube to the countertop or other undesired areas for toothpaste. Any toothpaste that is outside of the tube over an extended period of time becomes hard and is difficult to remove from counter tops and furnishings and can damage surface finishes. Hardened toothpaste makes replacing the threaded tube cap very difficult.

Currently, there are tubeless toothpaste products in the form of a compressed tablet. These products remove the need for a tube and help with the dexterity challenges often experienced by children and others. These tablet form tubeless products solve the mess problem and are easy to use for travel. Although the tablets solve these identified issues, they are not conducive for use by children. The compressed tablet form is quite dry in nature. As they are broken apart in the oral cavity, this dryness yields a chalky feel in the mouth. Children are more likely to swallow the tablet form because the tablet is not chewy and because of the chalky feel. Any user with propensity for dry mouth will also find the tablet form to be less satisfactory, as an abundance of saliva is needed to dissolve and distribute the dry tablet product.

Therefore, there is a need for a dental cleaning product which is not located in a tube. Further, there is a need for a dental cleaning product that can be used by those with dexterity problems, such as children. In addition, there is a need in the art for a dental cleaning product that comes in measured doses and is not dry and chalky.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a dental cleaning tablet. The dental cleaning tablet includes a core, wherein the core includes one or more dental hygiene products and an outer shell, wherein the outer shell completely covers the core.

Another example embodiment includes a dental cleaning tablet. The dental cleaning tablet includes a core. The core includes a sweetener, a surfactant, a humectant, an abrasive, a gelling agent, a microbial agent and a flavoring. The dental cleaning tablet also includes an outer shell. The outer shell is dry to the touch, completely covers the core and includes a sweetener and a sealing agent.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A is an outside view of a dental cleaning tablet;

FIG. 1B shows the internal elements of the dental cleaning tablet; and

FIG. 1C is a cutaway view of the dental cleaning tablet.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

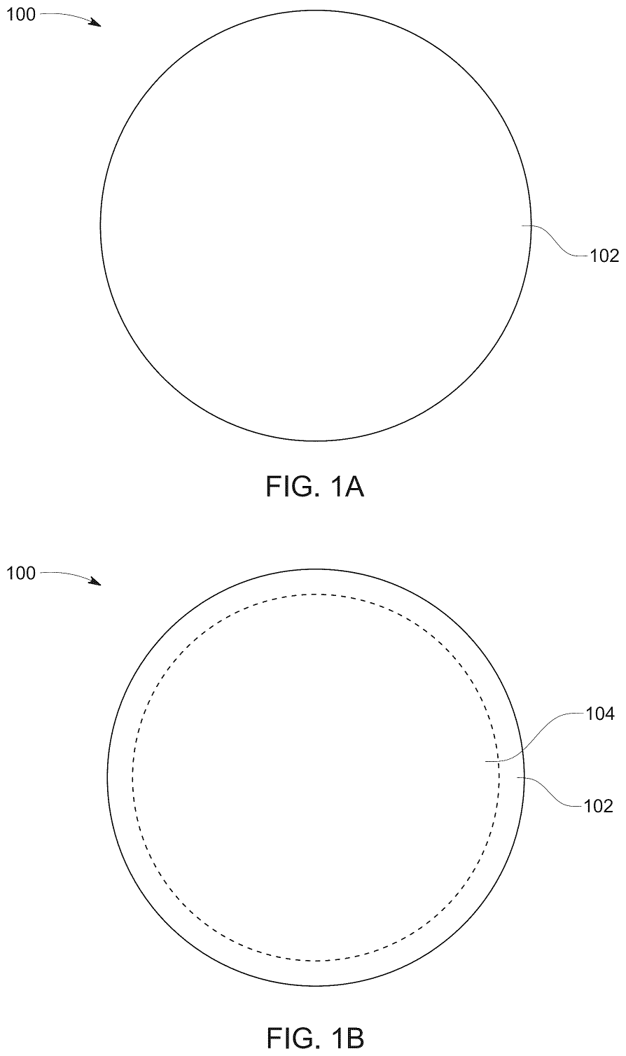

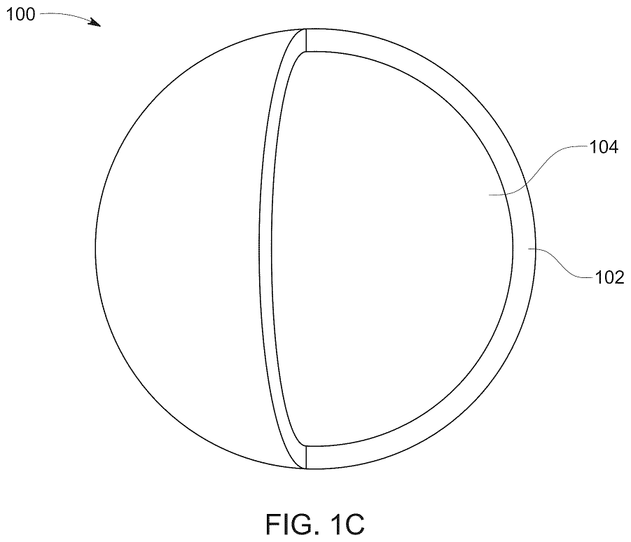

FIGS. 1A, 1B and 1C (collectively “FIG. 1”) illustrate an example of a dental cleaning tablet 100. FIG. 1A is an outside view of the dental cleaning tablet 100; FIG. 1B shows the internal elements of the dental cleaning tablet 100; and FIG. 1C is a cutaway view of the dental cleaning tablet 100. The dental cleaning tablet 100 configured to allow the user to easily brush their teeth and refresh their mouth. The dental cleaning tablet 100 provides a cleaning action for oral hygiene without the need for a toothpaste deposited from a tube. There is no waste because the dental cleaning tablet 100 is sized for a single use. While the dental cleaning tablet 100 is designed for use by children, it can be used by anyone. The compounds used in creating the dental cleaning tablet 100 are non-harmful, ingestible, and are typically used in other dental hygiene products. As used in the specification and the claims, the phrase “configured to” denotes an actual state of configuration that fundamentally ties recited elements to the physical characteristics of the recited structure. That is, the phrase “configured to” denotes that the element is structurally capable of performing the cited element but need not necessarily be doing so at any given time. Thus, the phrase “configured to” reaches well beyond merely describing functional language or intended use since the phrase actively recites an actual state of configuration.

The dental cleaning tablet 100 provides solutions to all the difficulties experienced by children and adults alike outlined above associated with the tooth brushing experience. Specifically, the dental cleaning tablet 100 provides the correct unit dose of toothpaste without the use of a tube.

The dental cleaning tablet 100 can be any desired shape. For example, shapes can include, but are not limited to, a sphere, a cube, a gum drop, a cylinder, a tablet, or any other shape that can fit conveniently inside the oral cavity for use. In general, spherical dental cleaning tablets 100 are easy to manufacture so spherical dental cleaning tablets 100 will be used as exemplary herein.

The dental cleaning tablet 100 is a premeasured amount of dental hygiene compounds which are easy to use. Therefore, it is critical that the dental cleaning tablet 100 has the appropriate volume. I.e., the intention is that when an individual is using the dental cleaning tablet 100 that it is the exact amount needed. Too little will mean that it is insufficient to clean the user's mouth. Too much will make the dental cleaning table 100 unwieldy and will create waste. Therefore, the volume of the dental cleaning tablet 100 is between 0.5 ml and 5 ml.

The dental cleaning tablet 100 reacts with the moisture in the mouth to to adhere cleaning ingredients to teeth using the toothbrush that allows for refreshing and thorough cleaning of teeth, mouth, and gums for efficient oral health.

FIG. 1 shows that the dental cleaning tablet includes an outer shell 102. When inserted into the mouth, the outer shell 102 rapidly dissolves. The outer shell 102 is dry to the touch and is chemically stable and sanitary, compared to conventional toothpastes. The outer shell 102 may be hard or soft to prevent damage during shipping and use (until the user wants to break the outer shell 102). The outer shell 102 mitigates the toothpaste mess by keeping non-hard materials (such as liquids and gels) contained until in the oral cavity. Further, the outer shell 102 means that the dental cleaning tablet 100 is easy to transport, easy to store without sticking to each other in the container and without the use of a toothpaste tube and easy to pick up for use.

In particular, the outer shell 102 is not messy to the touch. The dental cleaning tablet 100 can be handled with dry hands without a residue getting on the user's fingers. The outer shell 102 also makes it easy for children to pick up an appropriate does with a single hand. Because it does not require two hands to use, it is much easier for children and others with dexterity challenges to use.

The outer shell 102 can include any desired composition. For example, the outer shell 102 can include a sweetener and a sealing agent.

FIG. 1 also shows that the dental cleaning tablet 100 can include a core 104. The purpose of the core 104 is to provide a pleasant experience without the hard, crunchy, and chalky feeling afforded by hard chewable tablet products. The core 104 is formulated to dissolve within 60 seconds or less upon chewing and brushing. The adhesion action is important to get the active ingredients to stick to the teeth for as long as possible.

The core 104 must be sealed and polished. The outer shell 102 serves the purpose of sealing the core 104 to protect it from contact with other pieces and to keep moisture out. The outer shell 102 and the core 104 together promote a better toothbrushing experience. The core 104 promotes additional chewing and discourages immediate shallowing by children.

The core 104 can include sweeteners including one or more of the following:
Xylitol in concentrations of between 1% and 40% by weight;
maltodextrin in concentrations of between 2% and 60% by weight;
stevia in concentrations of between 2% and 60% by weight;
maltitol in concentrations of between 5% and 70% by weight;
sorbitol in concentrations of between 5% and 70% by weight;
erythritol in concentrations of between 1% and 20% by weight;
isomalt in concentrations of between 5% and 20% by weight;
mannitol in concentrations of between 5% and 20% by weight; or
other sugar alcohols in concentrations of between 2% and 70% by weight.

As used in the specification and the claims, the term approximately shall mean that the value is within 10% of the stated value, unless otherwise specified.

The core 104 can also include surfactants including one or more of the following:
Sodium Laureth Sulfate in concentrations of between 1% and 10% by weight;
Sodium Laurel Sulfocate in concentrations of between 1% and 10% by weight;
Cocamidopropyl betaine in concentrations of between 1% and 10% by weight;
Decyl glucoside in concentrations of between 1% and 10% by weight;
Sodium dodecyl sulfate in concentrations of between 1% and 10% by weight;
Polysorbate 20 in concentrations of between 1% and 10% by weight;
Lauryl glucoside in concentrations of between 1% and 10% by weight;
Polysorbate 80 in concentrations of between 1% and 10% by weight;
Nonionic surfactant in concentrations of between 1% and 10% by weight;
Sulfonate in concentrations of between 1% and 10% by weight;
Sodium stearate in concentrations of between 1% and 10% by weight;
Coco glucoside in concentrations of between 1% and 10% by weight;
Sodium lauroyl sarcosinate in concentrations of between 1% and 10% by weight;
Sodium cocoyl isethionate in concentrations of between 1% and 10% by weight;
Polyethylene glycol in concentrations of between 1% and 10% by weight;
Sorbitan monolaurate in concentrations of between 1% and 10% by weight;
Poloxamer in concentrations of between 1% and 10% by weight;
Surfactin in concentrations of between 1% and 10% by weight;
Sorbitan in concentrations of between 1% and 10% by weight;
Rhamnolipid in concentrations of between 1% and 10% by weight;
Glycerol monostearatej in concentrations of between 1% and 10% by weight;
Benzalkonium chloride in concentrations of between 1% and 10% by weight;
Cetyl alcohol in concentrations of between 1% and 10% by weight; or
Alkyl polyglycoside in concentrations of between 1% and 10% by weight.

The core 104 can further include humectants including one or more of the following:
Glycerin in concentrations of between 1% and 50% by weight;
Hyaluronic acid in concentrations of between 1% and 50% by weight;
Propylene glycol in concentrations of between 1% and 50% by weight;
Sorbitol in concentrations of between 1% and 50% by weight;
Aloe vera in concentrations of between 1% and 50% by weight;
Panthenol in concentrations of between 1% and 50% by weight;
Urea in concentrations of between 1% and 50% by weight;
Honey in concentrations of between 1% and 50% by weight; or
Polyglycitol in concentrations of between 1% and 50% by weight.

The core 104 can moreover include mild abrasives including one or more of the following:
Aluminum oxide in concentrations of between 0.5% and 10% by weight;
calcium pyrophosphate in concentrations of between 0.5% and 10% by weight;
silica in concentrations of between 0.5% and 10% by weight;
sodium bicarbonate in concentrations of between 0.1% and 10% by weight; or
Calcium carbonate in concentrations of between 0.5% and 10% by weight.

The core 104 can additionally include a remineralization agent including one or more of:
Fluoride in concentrations of between 0.5% and 20% by weight;
Nano-hydroxyapatite in concentrations of between 0.5% and 20% by weight;
Coconut Oil in concentrations of between 0.5% and 20% by weight; or
Xylitol in concentrations of between 0.5% and 20% by weight.

The core 104 can also include a gelling agent including one or more of:
Pectin in concentrations of between 0.5% and 10% by weight; or
Gelatin in concentrations of between 0.5% and 10% by weight.

The core 104 can further include an antimicrobial agent including one or more of:
Xylitol in concentrations of between 2% and 40% by weight;
Sodium Bicarbonate in concentrations of between 1% and 20% by weight;
Sorbitol in concentrations of between 2% and 20% by weight; or
Hydrogen peroxide in concentrations of between 0.1% and 10% by weight.

The core 104 can further include a flavoring including one or more of:
A fruit oil or essence in concentrations of between 0.01% and 5% by weight selected from at least one of:
   apple;
   apricot;
   banana;
   blueberry;
   cherry;
   cookie dough
   bubblegum;
   mint flavors;
   grape;
   grapefruit;
   kiwi;
   lemon;
   lime;
   orange;
   pear;
   peach;
   pineapple;
   plum;
   raspberry;
   strawberry;
   tangerine; and
   watermelon.

After insertion into the mouth, the outer core 102 dissolves or can be chewed to break open. Once the outer shell 102 is broken open, the user can begin brushing. This will activate dissolution of the core as it adheres to the teeth and gums. The user then brushes the deposited dilution. The correct balance of mild abrasives and humectants, and remineralizers is spread adequately around the oral cavity to clean and protect teeth from decay. The dental cleaning tablet 100 is specifically designed for children avoiding the need for teeth whiteners, gingivitis fighting ingredients, and other chemicals typically found in adult toothpastes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A dental cleaning tablet, the dental cleaning tablet comprising:
a core, wherein the core includes one or more dental hygiene products; and
an outer shell, wherein the outer shell completely covers the core.

2. The dental cleaning tablet of claim 1 wherein the outer shell includes:
a sweetener; and
a sealing agent.

3. The dental cleaning tablet of claim 1 further comprising:
a microbial agent, wherein the antimicrobial agent includes at least one of:
Xylitol;
Sodium Bicarbonate;
sorbitol; or
hydrogen peroxide.

4. The dental cleaning tablet of claim 1, wherein the outer shell is a sphere with diameter between 3 mm and 20 mm.

5. The dental cleaning tablet of claim 1, wherein the inner core includes a gummy material.

6. The dental cleaning tablet of claim 1, wherein the dental cleaning tablet is edible.

7. The dental cleaning tablet of claim 1, wherein the outer shell is dry to the touch.

8. The dental cleaning tablet of claim 1, wherein the core further includes one or more flavorings, wherein the one or more flavorings includes an essence selected from:
apple;
apricot;
banana;
blueberry;
cherry;
bubblegum;
cookie dough;
mint flavors;
grape;
grapefruit;
kiwi;
lemon;
lime;
orange;
pear;
peach;
pineapple;
plum;
raspberry;
strawberry;
tangerine; or
watermelon.

9. The dental cleaning tablet of claim 1, wherein the one or more dental hygiene products includes:
a mild abrasive;
a surfactant;
a gelling agent;
a humectant; and
a sweetener.

10. The dental cleaning tablet of claim 9, wherein the sweetener is present in a concentration from about 0.005% to about 80% by weight.

11. The dental cleaning tablet of claim 9, wherein the sweetener includes at least one of:
a non-caloric saccharin;
natural high-intensity sweeteners; or
sugar alcohols.

12. The dental cleaning tablet of claim 9, wherein the sweetener includes at least one of:
Xylitol;
maltodextrin;
stevia;
maltose;
maltitol;
sorbitol;
erythritol;
isomalt; or
mannitol.

13. The dental cleaning tablet of claim 9, wherein the surfactant includes at least one of:
Sodium Laureth Sulfate;
Sodium Laurel Sulfocate;
Cocamidopropyl betaine;
Decyl glucoside;
Sodium dodecyl sulfate;
Polysorbate 20;
Lauryl glucoside;
Polysorbate 80;
Nonionic surfactant;
Sulfonate;
Sodium stearate;
Coco glucoside;
Sodium lauroyl sarcosinate;
Sodium cocoyl isethionate;
Polyethylene glycol;
Sorbitan monolaurate;
Poloxamer;
Surfactin;
Sorbitan;
Rhamnolipid;
Glycerol monostearatej;
Benzalkonium chloride;
Cetyl alcohol; or
Alkyl polyglycoside.

14. The dental cleaning tablet of claim 9, wherein the humectant includes at least one of:
Glycerin;
Hyaluronic acid;
Propylene glycol;
Sorbitol;
Aloe vera;
Panthenol;
Urea; or
Honey.

15. The dental cleaning tablet of claim 9, wherein the mild abrasive includes at least one of:
Aluminum oxide;
calcium pyrophosphate;
silica; or
sodium bicarbonate
calcium carbonate
dicalcium phosphate.

16. The dental cleaning tablet of claim 9, further comprising a remineralization agent.

17. The dental cleaning tablet of claim 16 wherein the remineralization agent includes at least one of:
Fluoride;
Nano-hydroxyapatite;
Coconut Oil; or
Xylitol.

18. The dental cleaning tablet of claim 9, wherein the gelling agent includes at least one of:
Pectin; or
Gelatin.

19. A dental cleaning tablet comprising:
a core, wherein the core includes:
a sweetener;
a surfactant;
a humectant;
an abrasive;
a gelling agent;
a remineralization agent;
a microbial agent; and
a flavoring; and
an outer shell, wherein the outer shell:
is dry to the touch;
completely covers the core; and
includes:
a sweetener;
and a sealant agent.
